(19) United States
(12) Patent Application Publication
(10) Pub. No.: US 2011/0077579 A1
(43) Pub. Date: Mar. 31, 2011

(54) Cochlear implant with localized fluid transport

(76) Inventors: William V. Harrison, Valencia, CA (US); Thomas J. Lobl, Valencia, CA (US); Stephen J. McCormack, Claremont, CA (US)

(21) Appl. No.: 11/387,206

(22) Filed: Mar. 23, 2006

Related U.S. Application Data
(60) Provisional application No. 60/665,171, filed on Mar. 24, 2005.

(51) Int. Cl. A61F 11/04; A61N 1/05; A61N 1/36

(52) U.S. Cl. 604/20; 607/137

(57) ABSTRACT

An apparatus for providing fluid communication with a cochlear lumen, the apparatus includes a delivery-tube configured for insertion into the cochlear lumen. Delivery-tube orifices on the delivery-tube provide fluid communication between a lumen defined by the delivery-tube and the cochlear lumen.

### RELATED APPLICATIONS

This application claims priority under 35 U.S.C. §119 to U.S. Provisional Patent Application 60/665,171, filed Mar. 24, 2005, which is incorporated herein by reference.

### TECHNICAL FIELD OF DISCLOSURE

This disclosure is directed to cochlear implants, and in particular, to the delivery of drugs into the cochlea.

### BACKGROUND

Perception of sound begins when a sound wave strikes the eardrum, thereby causing it to vibrate. Vibration of the eardrum in turn causes vibration of small bones in the middle-ear, to which the eardrum is mechanically coupled. These bones transmit the energy from the sound wave into a fluid that fills the cochlea, thereby initiating a pressure wave that propagates through the fluid.

The pressure wave brushes past hairs that line the interior of the cochlea, setting those hairs into motion as it does so. These hairs are coupled to auditory nerves. Hence, stimulation of the hairs results in nerve stimulation. The extent to which the hairs are bent determines the loudness of the sound. The location of the hair within the cochlea determines the frequency, or pitch of the sound.

In certain diseases, the cochlea develops what amounts to bald spots. These bald spots result in loss of the ability to perceive those frequencies that correspond to the locations of those bald spots. Cochlear implants provide electrodes that mimic the function of those missing hairs by applying electric fields to stimulate selected portions of the cochlea in response to detected sound.

For cochlear implants to carry out their function more effectively, it is helpful for the electrodes to be close to the neural tissue that is to be stimulated. It is therefore desirable to stimulate growth of neural tissue toward the electrode. One way to stimulate neural-growth is to introduce neural-growth factors.

A difficulty, however, is that neural-growth factors are strong drugs with potentially significant side effects. Hence, it is undesirable to administer such drugs systemically.

### SUMMARY

The systems and techniques described here help facilitate fluid transport to or from selected local sites within the cochlea.

In one aspect, an apparatus for providing fluid communication with a cochlear lumen includes a delivery-tube defining a delivery-tube lumen configured for insertion into the cochlear lumen. The delivery-tube includes a plurality of delivery-tube orifices. These orifices provide fluid communication between the delivery-tube lumen and the cochlear lumen.

The apparatus optionally includes a pump coupled to the delivery-tube lumen to provide pump head pressure to fluid contained within the delivery-tube lumen. The pump head pressure can be such as to force fluid out of the delivery-tube through the orifices or to draw fluid into the delivery-tube through the orifices.

Where a pump is present, the apparatus can also include a reservoir in fluid communication with the pump. The pump is then configured to transfer delivery fluid between the delivery-tube lumen and the reservoir.

In some implementations, the apparatus further includes a number of valves, each of which is associated with an orifice. The valves are configured to control flow through the orifice, between the cochlear lumen and the delivery-tube lumen.

Other implementations may include electrodes disposed along a path following the delivery-tube, with each electrode being in electrical communication with the cochlear lumen. The electrodes can be coated with, for example, a titanium oxide coating.

Another aspect includes an apparatus for providing fluid communication with a cochlear lumen. Such an apparatus includes a carrier-tube that defines a carrier-tube lumen. The carrier-tube is configured to extend along the cochlear lumen. It includes carrier-tube orifices providing fluid communication with a carrier-tube lumen. An array of electrodes extends along the carrier-tube, each of the electrodes in the array being coated with a titanium oxide coat. A delivery-tube extends through the carrier-tube and defines a delivery-tube lumen. The delivery-tube includes delivery-tube orifices that provide fluid communication between the delivery-tube lumen and the cochlear lumen. A pump pumps fluid between a reservoir and the delivery-tube lumen. Valves, each associated with a delivery-tube orifice, regulate fluid flow between the delivery-tube lumen and the cochlear lumen. The valves, pump, and electrodes are all under control of a control system.

Another aspect features a method for improving coupling between a cochlear implant the neural tissue within the cochlea. The method includes selecting a plurality of locations on the neural tissue and locally administering a neural-growth controller to neural tissue at the selected locations.

Alternative practices of the method include those in which one selectively provides electrical stimulation to neural tissue at each of the locations.

In other implementations, locally administering neural-growth controller includes locally administering a neural-growth suppressant, or a neural-growth factor.

Additional implementations may include providing electrical stimulation and controlling that stimulation to encourage neural-growth toward selected electrodes.

These and other features and advantages will be apparent from the following detailed description and the accompanying figures, in which:

### BRIEF DESCRIPTION OF THE DRAWINGS

FIG. 1 shows a stimulation pulse;

FIG. 2A shows a cochlear stimulation system for delivering the stimulation pulse of FIG. 1;

FIG. 2B is a block diagram of the speech processor used in the system of FIG. 2A;

FIGS. 3A and 3B show portions of the electrode array deployed in the cochlea;

FIG. 4 shows a fluid transport system; and

FIG. 5 shows a fluid transport system for delivery of multiple delivery fluids.

### DETAILED DESCRIPTION

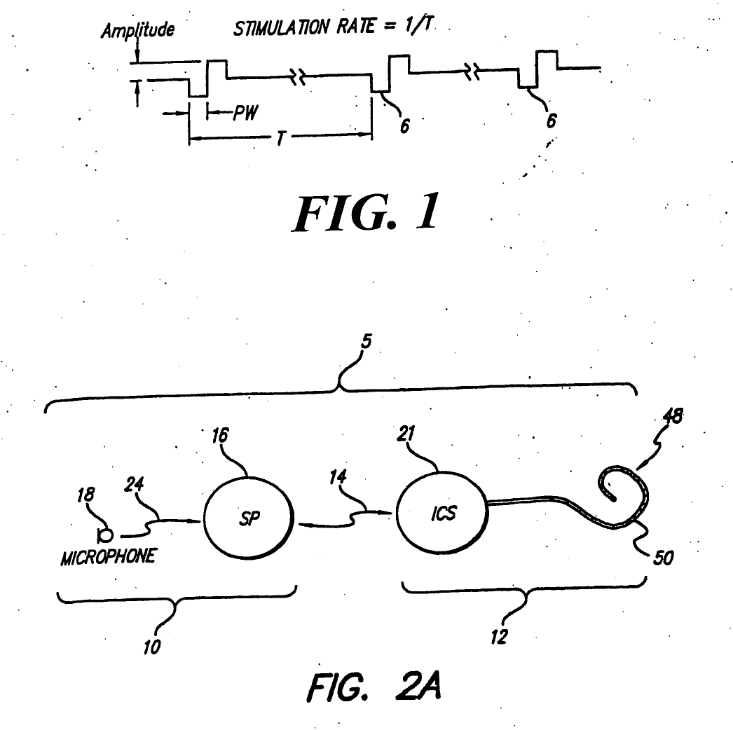

FIG. 1 shows a biphasic pulse train having a stimulation rate (1/T), pulse width and pulse amplitude as those terms are commonly used in connection with a neurostimulator device, such as a cochlear implant, a spinal cord stimulator, a deep brain stimulator, or other neural stimulator. All such systems commonly stimulate tissue with biphasic pulses 6 of the type shown in FIG. 1.

A “biphasic” pulse 6 consists of two pulses: a first pulse of one polarity having a specified magnitude, followed immediately, or shortly thereafter, by a second pulse of the opposite polarity, although possibly of different duration and amplitude. The amplitudes and durations are selected so that the total charge of the first pulse equals the total charge of the second pulse. Such charge-balancing is believed to reduce damage to stimulated tissue and to reduce electrode corrosion. For multi-channel cochlear stimulators, it is common to apply a high rate biphasic stimulation pulse train to each of the pairs of electrodes in the implant (described below) in accordance with a selected strategy and to modulate the pulse amplitude of the pulse train as a function of information contained within a feedback acoustic signal.

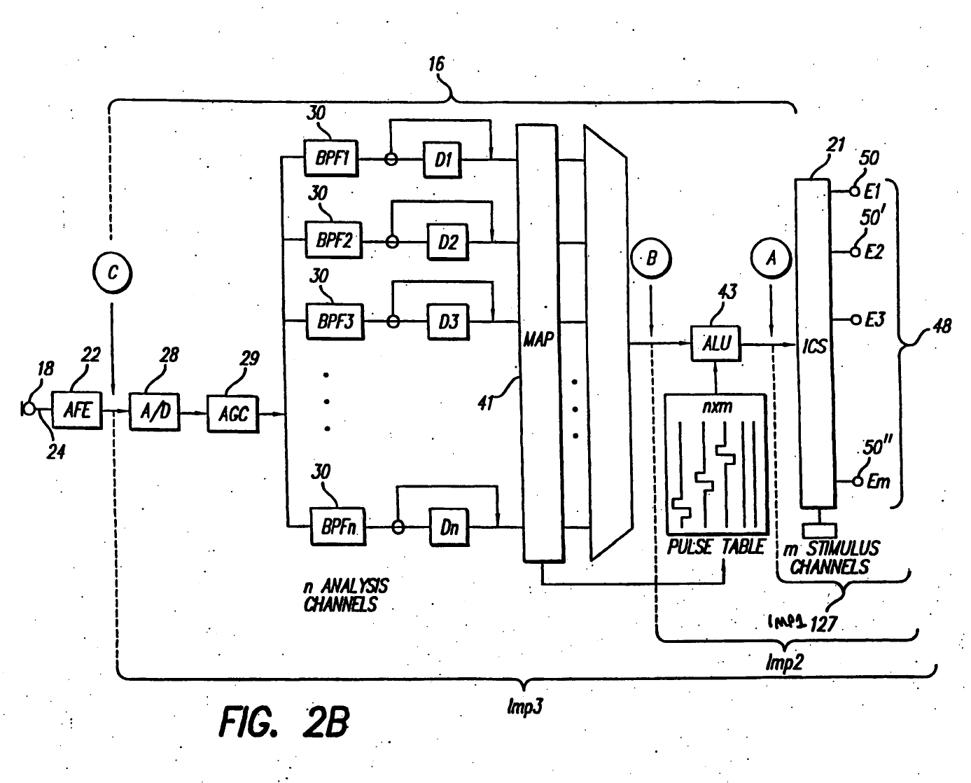

A cochlear stimulation system 5, as shown in FIG. 2A, includes a speech processor portion 10 and a cochlear stimulation portion 12. The speech-processor portion 10 includes a speech processor 16 and a microphone 18. The microphone 18 may be connected directly to the speech processor 16 or coupled to the speech processor 16 through an appropriate communication link 24. The cochlear-stimulation portion 12 includes an implantable cochlear-stimulator 21 and an electrode array 48 adapted for insertion within the cochlea of a patient. The array 48 includes a plurality of electrodes 50 spaced along the array length. These electrodes 50 are selectively connected to the implantable cochlear-stimulator 21. In typical embodiments, there are sixteen electrodes 50, however there exist embodiments with as few as four to as many as sixty-four electrodes 50. Each electrode 50 in the array is a platinum-iridium electrode.

Typical electrode arrays 48 include those described in U.S. Pat. Nos. 4,819,647 or 6,129,753, both of which are incorporated herein by reference. Electronic circuitry within the implantable cochlear-stimulator 21 allows a specified stimulation current to be applied to selected pairs, or groups, of the individual electrodes 50 within the electrode array 48 in accordance with a specified stimulation pattern defined by the speech processor 16.

The implantable cochlear-stimulator 21 and the speech processor 16 are linked by a suitable data or communications link 14. In some cochlear implant systems, the speech processor 16 and microphone 18 comprise the external portion of the cochlear implant system and the implantable cochlear-stimulator 21 and electrode array 48 comprise the implantable portion of the system. In such cases, the data link 14 is a transcutaneous data link that allows power and control signals to be sent from the speech processor 16 to the implantable cochlear-stimulator 21. In some embodiments, data and status signals may also be sent from the implantable cochlear-stimulator 21 to the speech processor 16.

Certain portions of the cochlear stimulation system 5 can be contained in a behind-the-ear unit that is positioned at or near the patient's ear. For example, the behind-the-ear unit can include the speech processor 16 and a battery module, both of which are coupled to a corresponding implantable cochlear-stimulator 21 and an electrode array 48. A pair of behind-the-ear units and corresponding implants can be communicatively linked via a Bionet System and synchronized to enable bilateral speech information conveyed to the brain via both the right and left auditory nerve pathways. The Bionet system uses an adapter module that allows two behind-the-ear units to be synchronized both temporally and tonotopically to maximize a patient's listening experience.

FIG. 2B shows a partial block diagram of one embodiment of a cochlear implant system capable of providing a high pulsatile stimulation pattern to virtual electrodes by appropriately weighting stimuli applied to real electrodes 50. At least certain portions of the speech processor 16 can be included within the implantable portion of the overall cochlear implant system, while other portions of the speech processor 16 can remain in the external portion of the system. In general, at least the microphone 18 and associated analog-front-end (“AFE”) circuitry 22 can be part of the external portion of the system and at least the implantable cochlear-stimulator 21 and electrode array 48 can be part of the implantable portion of the system. As used herein, the term “external” means not implanted under the skin or residing within the inner ear. However, the term “external” can also mean residing within the outer ear, residing within the ear canal or being located within the middle ear.

Typically, a transcutaneous data link between the external portion and implantable portions of the system is implemented by using an internal antenna coil within the implantable portion, and an external antenna coil within the external portion. In operation, the external antenna coil is aligned over the location at which the internal antenna coil is implanted, thereby inductively coupling the coils to each other. This allows data (e.g., the magnitude and polarity of a sensed acoustic signals) and power to be transmitted from the external portion to the implantable portion.

In other embodiments, both the speech processor 16 and the implantable cochlear-stimulator 21 may be implanted within the patient, either in the same housing or in separate housings. If the speech processor 16 and the stimulator 21 are in the same housing, the link 14 may be implemented with a direct wire connection within the housing. If the speech processor 16 and stimulator 21 are in separate housings, as described, in U.S. Pat. No. 6,067,474, the contents of which are herein incorporated by reference, the link 14 may be an inductive link using a coil or a wire loop coupled to the respective parts.

The microphone 18 converts incident sound waves into corresponding electrical signals. The electrical signals are sent to the speech processor 16 over a suitable electrical or other link 24. The speech processor 16 processes these signals in accordance with a selected speech processing strategy to generate appropriate control signals for controlling the implantable cochlear-stimulator 21. Such control signals specify the polarity, magnitude, which electrode pair or electrode group is to receive the stimulation current, and when each electrode pair is to be stimulated. Such control signals thus combine to produce a desired time-varying electric field distribution in accordance with a desired speech processing strategy.

A speech processing strategy conditions the magnitude and polarity of the stimulation current applied to the implanted electrodes of the electrode array 48. Such a speech processing strategy involves defining a pattern of stimulation waveforms that are to be applied to the electrodes as controlled electrical currents.

FIG. 2B depicts the functions that are carried out by the speech processor 16 and the implantable cochlear-stimulator 21. It should be appreciated that the functions shown in FIG. 2B (dividing the incoming signal into frequency bands and independently processing each band) are representative of just one type of signal processing strategy that may be employed. Other signal processing strategies could just as easily be used to process the incoming acoustical signal. A description of the functional block diagram of the cochlear implant shown in FIG. 2B is found in U.S. Pat. No. 6,219,580, the contents of which are incorporated herein by reference. The system and method described herein may be used with cochlear systems other than the system shown in FIG. 2B.

The cochlear implant functionally shown in FIG. 2B provides n analysis channels that may be mapped to one or more stimulus channels. That is, after the incoming sound signal is received through the microphone 18 and the analog front end circuitry (AFE) 22, the signal can be digitized in an analog-to-digital (A/D) converter 28 and then subjected to appropriate gain control (which may include compression) in an automatic gain control (AGC) unit 29.

After appropriate gain control, the signal can be divided into n analysis channels 30, each of which includes at least one bandpass filter, BPFn, centered at a selected frequency. The signal present in each analysis channel 30 is processed as described more fully in the U.S. Pat. No. 6,219,580 patent, or as is appropriate, using other signal processing techniques. Signals from each analysis channel may then be mapped, using a mapping function 41, so that an appropriate stimulus current of a desired amplitude, polarity, and timing may be applied through a selected stimulus channel to stimulate the auditory nerve.

The exemplary system of FIG. 2B provides n analysis channels for analysis of an incoming signal. The information contained in these n analysis channels is then appropriately processed, compressed and mapped to control the actual stimulus patterns that are applied to the user by the implantable cochlear-stimulator 21 and its associated electrode array 48.

The electrode array 48 includes a plurality of electrode contacts 50, 50′ 50″ and labeled as E1, E2, . . . Em, respectively, which are connected through appropriate conductors to respective current generators or pulse generators within the implantable cochlear-stimulator. These electrode contacts define m stimulus channels 127 through which individual electrical stimuli can be applied at m different stimulation sites within the patient's cochlea or other tissue stimulation site.

It is common to use a one-to-one mapping scheme between the n analysis channels and the m stimulus channels 127 that are directly linked to m electrodes 50, 50′, 50″, such that n analysis channels=m electrodes. In such a case, the signal resulting from analysis in the first analysis channel may be mapped, using appropriate mapping circuitry 41 or equivalent, to the first stimulation channel via a first map link, resulting in a first cochlear stimulation site (or first electrode). Similarly, the signal resulting from analysis in the second analysis channel of the speech processor may be mapped to a second stimulation channel via a second map link, resulting in a second cochlear stimulation site, and so on.

In some instances, a different mapping scheme may prove beneficial. For example, assume that n is not equal to m (n, for example, could be at least 20 or as high as 32, while m may be no greater than sixteen, e.g., 8 to 16). The signal resulting from analysis in the first analysis channel may be mapped, using appropriate mapping circuitry 41 or equivalent, to the first stimulation channel via a first map link. This results in a first stimulation site (or first area of neural excitation). Similarly, the signal resulting from analysis in the second analysis channel of the speech processor may be mapped to the second stimulation channel via a second map link. This results in a second stimulation site. Also, the signal resulting from analysis in the second analysis channel may be jointly mapped to the first and second stimulation channels via a joint map link. This joint link results in a stimulation site that is somewhere in between the first and second stimulation sites.

The “in-between” site at which a stimulus is applied may be viewed as a “stimulation site” produced by a virtual electrode. Advantageously, this capability of using different mapping schemes between n speech processor analysis channels and m implantable cochlear-stimulator stimulation channels to thereby produce stimulation sites corresponding to virtual electrodes provides a great deal of flexibility in positioning the neural excitation areas precisely in the cochlea.

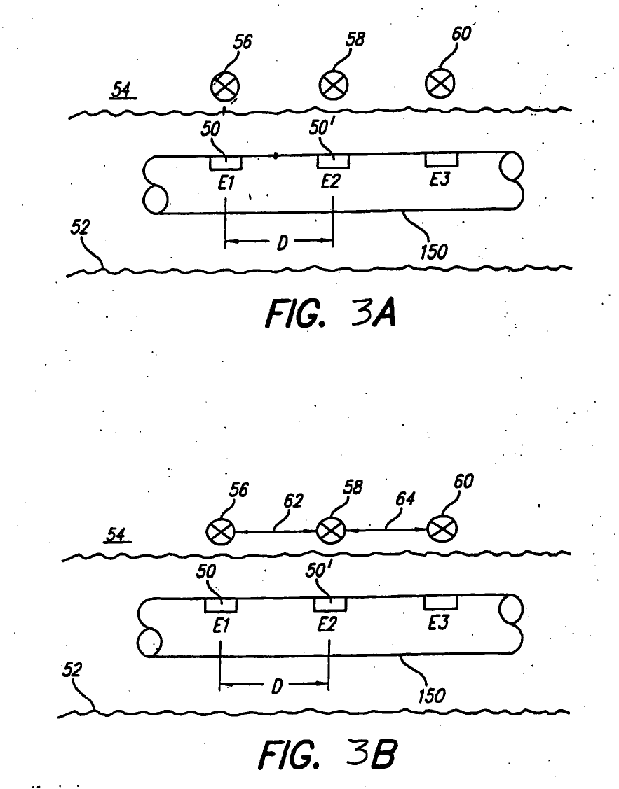

As explained in more detail below in connection with FIGS. 3A and 3B, through appropriate weighting and sharing of currents between two or more physical electrodes, it is possible to provide a large number of virtual electrodes between physical electrodes, thereby effectively steering the location at which a stimulus is applied to almost any location along the length of the electrode array.

An exemplary output stage of the implantable cochlear-stimulator 21, which connects with each electrode E1, E2, E3, . . . Em of the electrode array, is described in U.S. Pat. No. 6,181,969, the contents of which are incorporated herein by reference. Such an output stage advantageously provides a programmable N-DAC or P-DAC (where DAC stands for digital-to-analog converter) connected to each electrode so that a programmed current may be sourced to or sunk from the electrode. Such a configuration permits pairing any electrode with any other electrode and adjusting the complex amplitudes of the currents to gradually shift the stimulating current that flows from one electrode, through the tissue, to another adjacent electrode or electrodes. This enables one to gradually shift the current from one or more electrodes to another electrode(s). Through such current shifting, the stimulus current may be shifted or directed so that it appears to the tissue that the current is coming from or going to an almost infinite number of locations.

FIG. 3A illustrates stimulus location when virtual electrodes are employed. In FIG. 3A, three electrodes E1, E2 and E3 of an electrode array are illustrated. A reference electrode, not shown, is present some distance from the electrodes E1, E2 and E3, thereby allowing monopolar stimulation to occur between a selected one of the electrodes and the reference electrode. Bipolar stimulation could likewise occur, e.g., between electrodes E1 and E2, between E2 and E3, or between any other pair of electrodes.

The electrodes E1, E2 and E3 are located “in line” on a carrier 150, and are spaced apart from each other by a distance “D.” Each electrode is electrically connected to the implantable cochlear-stimulator 21 by a wire conductor (not shown) embedded within the carrier 150. The carrier 150 is shown inserted through a duct 52 adjacent to the tissue 54 that is to be stimulated. For a cochlear implant system, the duct 52 typically comprises the scala tympani of a human cochlea.

When a stimulus current is applied to electrode E1, the stimulus location in the tissue 54 is essentially the location 56, adjacent to the physical location of the electrode E1. Similarly, when a stimulus current is applied to electrode E2, the stimulus location in the tissue 54 is essentially the location 58, adjacent to the physical location of the electrode E2. Likewise, when a stimulus current is applied to electrode E3, the stimulus location in the tissue 54 is essentially the location 60, adjacent to the physical location of the electrode E3. It is thus seen that the resolution, or precision, with which a stimulus may be applied to the tissue is only as good as is the spacing of the electrodes 50 on the electrode array. That is, each stimulus location in the tissue 54 is separated by approximately the same distance “D” that separates the electrodes 50.

FIG. 3B shows the location of a stimulus when current steering creates virtual electrodes. The structure of the electrode array and spacing between electrodes E1, E2 and E3 is the same as that shown in FIG. 3A. Thus, when a stimulus current is applied only to the electrode E1, the stimulus location in the tissue 54 is the same location 56 as was the case in FIG. 3A. Similarly, when a stimulus current is applied only to the electrode E2, the stimulus location in the tissue 54 is the location 58. Likewise, when a stimulus current is applied only to electrode E3, a stimulus location in the tissue 54 is the location 60. However, through application of current steering, a stimulus current may be shared, e.g., between electrodes E1 and E2 (and some other paired or reference electrode). This sharing permits the stimulus location to be placed anywhere along the line 62 between points 56 and 58. Alternatively, if the current is shared between electrodes E2 and E3, the location in the tissue where the stimulus is directed may be anywhere along the line 64 between points 58 and 60.

To illustrate further, suppose a stimulus current having an amplitude I1 is applied to the tissue through electrode E1 (and some reference electrode). The location within the tissue 54 where the stimulus would be felt would be the point 56. However, if a stimulus current of only 0.9×I1 were applied through electrode E1 at the same time that a stimulus current of 0.1×I1 were applied through electrode E2, then the stimulus location within the tissue 54 would be a little to the right of the point 56. If the stimulus current applied through electrode E1 continued to be decreased while, at the same time, the current applied through electrode E2 were increased, then the stimulus location would move along the line 62 from left to right, i.e., from point 56 to point 58.

Similarly, by concurrently delivering current stimuli at electrodes E2 and E3, the location at which the effective stimulus would be felt would lie somewhere along the line 64, depending on the weighting of stimulus currents delivered at the two electrodes. This concept of current steering is described more fully in U.S. Pat. No. 6,393,325, incorporated herein by reference.

One method of implementing virtual electrodes is by concurrently delivering stimuli at two or more electrodes. Another way of implementing virtual electrodes is to present alternating stimuli at two electrodes in a time-multiplexed manner. For example, a first stimulus current is presented at the first electrode, then a second stimulus current is presented at the second electrode, then, the first stimulus current is presented at the first electrode, then the second stimulus current is presented at the second electrode, and so on, in a time multiplexed sequence. The first and second stimulus signals are usually different, e.g., they have different amplitudes and pulse widths. Such delivery of stimulation will be perceived as if a virtual electrode were delivering a stimulus, with the virtual electrode appearing to be located between the two physical electrodes.

The electrodes 50 shown in FIG. 3A are located at some distance from the tissue 54 that is to be stimulated. To enable the electrodes 50 to more efficiently stimulate the tissue 54, it is desirable to selectively grow the tissue 54 toward the electrodes 50. Certain drugs such as neural-growth factors, neurotrophins, neural-growth retardants, and vectoring agents, are known to stimulate and/or suppress growth of neural tissue 54. Such drugs, collectively referred to herein as “neural-growth controllers,” are often quite powerful and may have undesirable side effects. As a result, it is undesirable to deliver them systemically.

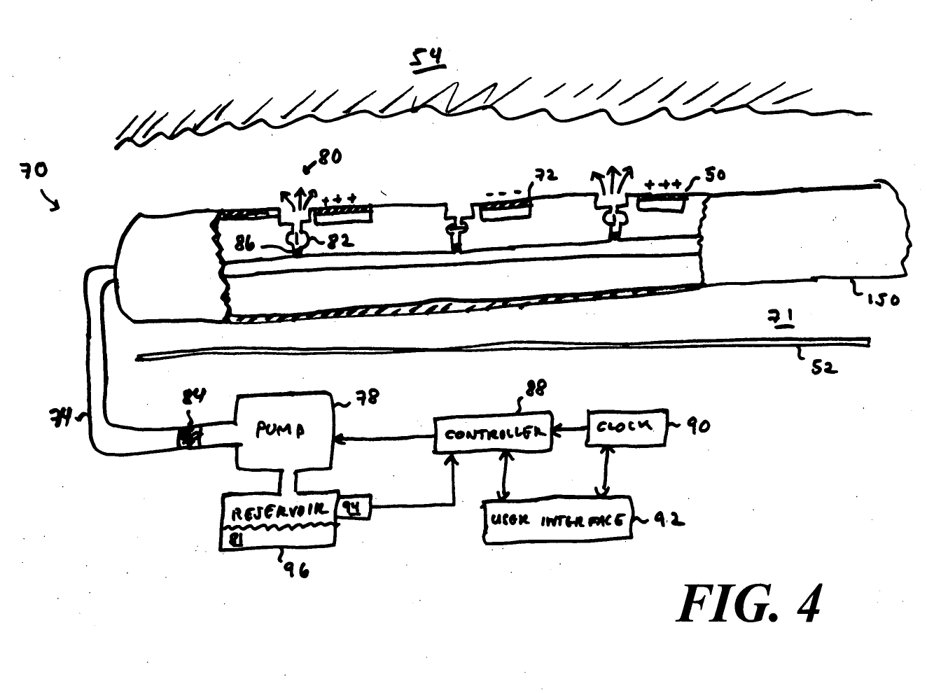

Local delivery of such neural-growth controllers into a lumen 71 of the duct 52 is achieved in an alternative embodiment, shown in FIG. 4, that incorporates a fluid transport system 70 for selectively delivering neural-growth controllers to regions surrounding particular electrodes 50. To further enhance their ability to stimulate and control growth of neural tissue 54, the electrodes 50 are coated with a layer 72 of titanium oxide.

The fluid transport system 70 includes a delivery-tube 74 that extends through a carrier lumen 76 in the carrier 150. The delivery-tube 74, which has an inner diameter between 0.02 microns and 10 microns, extends from a pump 78 to a distal end of the carrier 150. The pump 78 is connected to a reservoir 96 of delivery fluid 81. The reservoir 96 is preferably external so that it can readily be refilled or drained as needed.

An array of orifices 80, each of which is associated with an electrode 50, extends along the delivery-tube 74. Each orifice 80 provides a passage for delivery fluid 81 into the duct lumen 71 in the vicinity of an electrode 50. A valve 82 associated with each orifice 80 regulates passage of delivery fluid 81 between the delivery-tube 74 and the duct lumen 71.

To reduce the likelihood of contamination, the fluid transport system 70 includes an inlet filter 84 that filters the delivery fluid 81 before it enters the delivery-tube 74. Alternatively or conjunctively, the fluid transport system 70 features orifice filters 86 that filter the delivery fluid 81 as it enters the duct lumen 71.

A controller 88 located either in an implanted pump housing (not shown) or in a cochlear-implant housing (not shown) controls the pump 78 and the valves 82. The connections between the pump 78 and the individual valves 82 are omitted for clarity in FIG. 4. The controller 88 is typically a microcontroller that executes software for starting and stopping the pump 78, setting the head pressure, and opening and closing the valves 82. A clock 90 in data communication with the controller 88 is useful for scheduling in cases where delivery fluid 81 is to be pumped periodically, or cases in which the dose profile is expected to vary over time.

A programming interface 92 is also included to enable changes to the software executing on the controller 88 to accommodate changes in dosage. Preferably, the interface 92 is a wireless interface so that the controller 88 can be more easily programmed.

An optional sensor 94 provides the controller 88 with data indicative of the level of delivery fluid 81. However, the controller 88 can also estimate a remaining quantity of delivery fluid 81 on the basis of a starting quantity suitably decremented by data derived from a dosage history.

The delivery fluid 81 can be a neural-growth controller. However any drug that is to be locally administered can be placed in the reservoir 96. For example, the delivery fluid 81 can be an anti-inflammatory drug or an anti-infection agent. In addition, the delivery fluid 81 can be a diagnostic agent to assist in visualizing the inner ear. Such diagnostic agents include contrast-imaging agents, and light emitting or fluorescent agents to aid in visualizing the inner ear.

The pump 78 can be configured to apply either positive or negative head (pressure). When configured to apply positive head, the pump 78 draws fluid out of the reservoir 96 and pumps it into the duct lumen 71. When configured to apply negative head, the pump 78 draws fluid, for example, cochlear endolymph, out of the inner ear and deposits it into the reservoir 96. This ability to operate the pump 78 in both directions enables the system to both locally administer drugs and to locally withdraw samples.

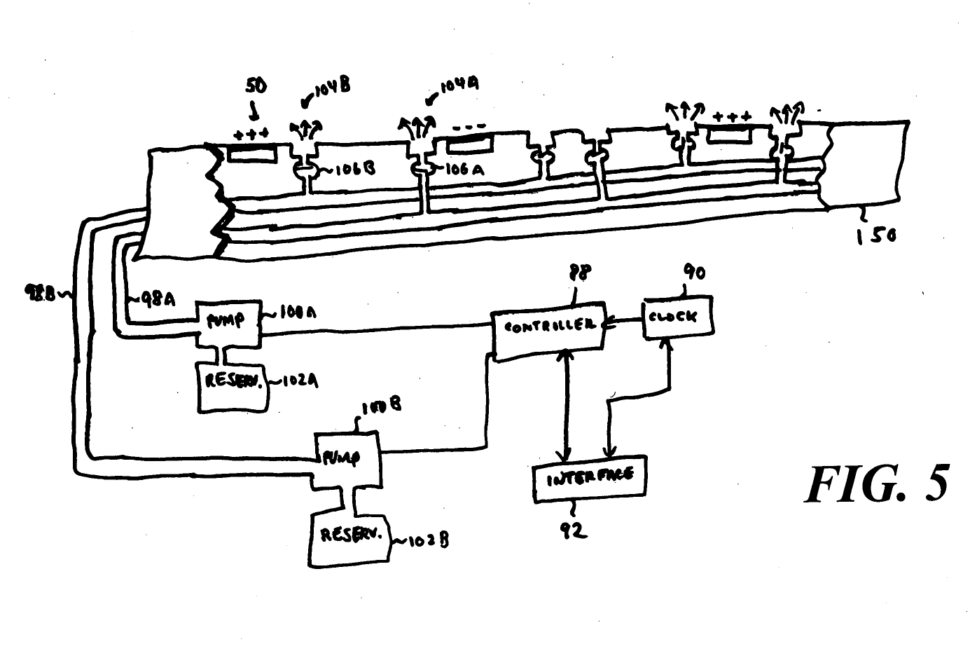

Another configuration, shown in FIG. 5, includes two delivery-tubes 98A, 98B extending through the carrier 150, each of which extends from corresponding pumps 100A, 100B controlled by a controller 88. Each pump 100A, 100B is connected to an associated reservoir 102A, 102B. Each delivery-tube 98A, 98B has orifices 104A, 104B associated with each electrode 50, and valves 106A, 106B to selectively open and close each orifice 104A, 104B. The configuration shown in FIG. 5 can readily be extended to include three or more delivery-tubes in communication with a corresponding number of reservoirs.

The controller 88 is programmed to open and close selected valves 82 at selected times and to operate the pump 78 at selected times and with selected heads. This enables the controller 88 to precisely meter out appropriate quantities of delivery fluid 81. In the case of the configuration shown in FIG. 5, the controller 88 can meter out appropriate quantities of different delivery fluids.

By appropriately controlling the pumps and valves, the controller 88 can create “virtual orifices” in the same manner discussed above in connection with virtual electrodes. The mathematical principles are the same, with the main difference being that the flow is that of a fluid instead of charge. In embodiments in which the pump 78 is reversible, the controller can operate the pump 78 in alternate directions. The controller 88 could thus more precisely meter dosage by, for example, causing the pump to “inhale” and “exhale” through a selected orifice. Or, by appropriately coordinating the pumping direction with the opening and closing of valves 82, the controller 88 can cause delivery fluid to be “exhaled” out one orifice and then “inhaled” through one or more adjacent orifices, thereby enabling precise placement of delivery fluid.

In the embodiment shown in FIG. 5, virtual orifices can be created for each delivery fluid independently by driving the pumps 100A, 100B associated with each delivery fluid and opening and closing corresponding valves 106A, 106B.

Some embodiments of the structure shown in FIG. 4 omit the valves 82 entirely. In these valveless embodiments, the pump 78 draws or pushes delivery fluid 81 through the orifices. The absence of valves 82 makes it difficult to control the spatial distribution of delivery fluid 81 with precision, however the temporal distribution can readily be controlled by controlling the head (including the sign thereof) provided by the pump 78.

Other embodiments omit the pump 78. In these embodiments, the flow of delivery fluid 81 is passive. In such a case, flow of delivery fluid would be driven by local variations in the ambient pressure within the duct lumen 71.

Some embodiments of the structure shown in FIG. 5 omit the valves the valves 106A, 106B entirely. In these embodiments, the pumps 100A, 100B draw or push the fluids contained in reservoirs 102A, 102B through the orifices 104A, 104B. The absence of valves 106A, 106B makes it difficult to control the spatial distribution of the fluids held in reservoirs 102A, 102B with precision, however the temporal distribution can readily be controlled by controlling the head (including the sign thereof) provided by the pumps 100A, 100B.

Other embodiments omit the pumps 100A, 100B. In these embodiments, the flow the fluids held in reservoirs 102A, 102B is passive. In such a case, flow of delivery fluid would be driven by local variations in the ambient pressure within the duct lumen 71.

The fluid transport systems 70 shown in FIGS. 4 and 5 enable neural-growth controllers to be delivered directly to the sites at which they are required and to be delivered in precisely calibrated doses with selected dose profiles according to a particular schedule.

By controlling the spatial distribution in which neural-growth controllers are delivered, one can stimulate neural-growth toward selected electrodes 50 and suppress neural-growth toward other electrodes 50. This results in neural-growth that embodies some tonotopic organization rather than the chaotic jumble of neural tissue 54 that may otherwise result.

Since drugs are delivered through orifices 80 adjacent to electrodes 50, it is possible to generate local electric fields before, during, or after the local administration of a drug. This ability to provide cooperative interaction between locally-applied electric fields and the locally-administered drug is believed to accelerate changes in the spatial distribution of neural tissue 54 and to enable more precise control over the tonotopic organization of such tissue 54.

The delivery-tube 74 can be either permanently within the carrier 150, or it can be withdrawn when no longer needed. Alternatively, the carrier 150 need not include electrodes 50 at all. The carrier 150 can be used solely to accommodate one or more delivery-tubes 74, without an array of electrodes 50.

The delivery-tube 74 or the carrier 150 can be coated with a polymer impregnated with a suitable drug. This embodiment is particularly useful for providing a bolus of a drug following implantation. Such a polymer-coated structure may be useful for introducing a bolus of anti-inflammatory agent or an anti-bacterial agent immediately following implantation.

In the case of a coated delivery-tube 74, the distribution of the drug along the length of the tube can vary with length. This results in regions of high drug concentration adjacent to regions of lower concentration. A delivery-tube coated in this way can locally deliver a bolus of a drug to selected portions of the duct lumen 71.

If no delivery fluid 81 is required, the delivery-tube can be replaced by a filament coated with a drug-impregnated polymer, as discussed above.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-6. (canceled)

7. An apparatus for providing fluid communication with a cochlear lumen, the apparatus comprising:
a delivery tube extending through a carrier, the carrier configured to extend along the cochlear lumen, the carrier having walls that define orifices;
a plurality of electrodes extending along the carrier;
a reservoir;
a pump for pumping fluid between the reservoir and the delivery tube;
a plurality of valves, each associated with an orifice, for controlling fluid flow between the orifices and the cochlear lumen, wherein each electrode is associated with only one of the orifices; and
a control system for controlling the pump, the valves, and the electrodes, wherein the control system is configured to independently control each valve.

8-12. (canceled)

13. The apparatus of claim 7, wherein the electrodes are each coated with a titanium oxide coating.

14-17. (canceled)

18. An apparatus for providing fluid and electrical communication with a cochlear lumen, the apparatus comprising:
a carrier configured for insertion into the cochlear lumen, the carrier comprising a pluralities of orifices;
a plurality of valves, each valve being independently controllable to control fluid flow between an orifice and the cochlear lumen; and
a plurality of electrodes extending along the carrier configured for electrical communication with the cochlear lumen, wherein each electrode is associated with only one of the orifices.

19. The apparatus of claim 18, further comprising a pump coupled to the delivery tube to provide pressure to the fluid within the carrier.

20. The apparatus of claim 19, further comprising a control system for controlling the pump, the valves, and the electrodes.

21. The apparatus of claim 19, further comprising a reservoir in fluid communication with the pump for providing the fluid to the carrier.

22. The apparatus of claim 18, wherein the electrodes are each coated with a titanium oxide coating.

23-24. (canceled)

25. The apparatus of claim 7, further comprising a filter located in a fluid flow path from the reservoir to at least one of the orifices.

26. The apparatus of claim 21, further comprising a filter located in a fluid flow path from the reservoir to at least one of the orifices.

27. The apparatus of claim 7, wherein the fluid comprises a neural growth controller.

28. The apparatus of claim 18, wherein the fluid comprises a neural growth controller.
